(19) United States
(12) Patent Application Publication
(10) Pub. No.: US 2004/0258640 A1
(43) Pub. Date: Dec. 23, 2004

(54) Interference pigment and compositions including same

(75) Inventor: Jean-Christophe Simon, Alsbach-Hahnlein (DE)

Correspondence Address: LERNER, DAVID, LITTENBERG,, KRUMHOLZ & MENTLIK, 600 SOUTH AVENUE WEST, WESTFIELD, NJ, 07090, US

(73) Assignee: L'OREAL, Paris (FR)

(21) Appl. No.: 10/664,197

(22) Filed: Sep. 17, 2003

(30) Foreign Application Priority Data
Sep. 18, 2002 (FR) 02 11579

(51) Int. Cl. A61K 7/021

(52) U.S. Cl. 424/63; 428/403; 106/401; 106/403; 106/489

(57) ABSTRACT

The present invention relates to an interference pigment having a multilayer structure, characterized in that it has a spectral reflectance that differs by not more than 20% from a reference spectral reflectance, preferably of a type of keratin material, for at least a portion of the visible spectrum 200 nm broad, this pigment being characterized in that the multilayer structure comprises at least one layer substantially totally coating an underlying layer.

### BACKGROUND OF THE INVENTION

The present invention relates to cosmetic or care compositions intended to be applied to the skin, including mucous membranes, and the integuments.

International patent application WO 00/75240 discloses interference pigments with a multilayer structure, having a spectral reflectance close to a reference spectral reflectance corresponding to a predetermined type of skin, for at least a portion of the visible spectrum.

These pigments may be obtained by depositing onto a polymer that is soluble in a given solvent, present on a flexible support, the successive layers of the multilayer structure, by means of a vacuum deposition technique. Next, the multilayer film is fragmented to form small platelets constituting the particles of the pigment.

Such pigments make it possible to produce compositions that imitate the colour of the skin, for example in order to cover it and mask its defects.

However, the platelets have a shape that promotes the specular reflection of light and their gloss must be attenuated by introducing a matting filler into the composition.

### SUMMARY OF THE INVENTION

The invention is especially directed towards further improving the pigments described in WO 00/75240 and in producing cosmetic or care compositions that allow, for example, an optimum imitation of the appearance of the skin or the integuments, for example in order to correct their defects, especially inhomogeneities of colour, or that allow a dominant undesirable chromatic to be attenuated.

According to one of its aspects, one subject of the invention is an interference pigment with a multilayer structure, this interference pigment having a spectral reflectance that differs by not more than 20% from the spectral reflectance of a type of keratin material for at least a portion of the visible spectrum 200 nm broad, this interference pigment being able to be characterized in that the multilayer structure comprises at least one layer substantially totally coating an underlying layer.

Such a interference pigment differs from the pigments obtained according to the method recalled above and described in WO 00/75240, for which the various layers do not coat the underlying layers in the region of the zones of rupture of the multilayer film that led to the formation of the platelets.

The invention especially makes it possible to produce interference pigment particles with a more “rounded” shape more easily, which can reduce their aptitude to specularly reflect light and can make them more matt, and thus allows them, for example, to recreate the appearance of the skin more faithfully.

In one working example of the invention, the interference pigment particles each have a general globular shape, especially a spherical shape. The interference pigment particles may, but need not comprise a substrate and that substrate, if used, may itself have a globular shape, especially a spherical shape, on which are deposited the various layers of the multilayer structure. The substrate may especially comprise a microsphere of an organic or mineral material, for example a glass or a metal, especially aluminium.

The substrate may be hollow or solid, and advantageously has a symmetrical shape relative to a centre of symmetry, for example a spherical or polyhedral shape.

The keratin material may be, for example, a skin, especially a Caucasian, half-caste, Asiatic or black skin.

In one working example of the invention, the spectral reflectance of the interference pigment is close to the spectral reflectance of the type of keratin material under consideration over the entire visible spectrum, i.e. over the wavelength range extending from 400 nm to 700 nm, which is 300 nm broad (a range which is 300 nm wide).

The spectral reflectance of the multilayer interference pigment may differ, for a range of the spectrum under consideration, for example for at least one range 200 nm broad, for example for at least the range extending from 500 nm to 700 nm, by not more than 10% of the spectral reflectance of the type of keratin material, or even not more than 5%, or of not more than 2%.

Preferably, the multilayer interference pigment is configured so as to have a lightness value L*, measured in the CIE 1976 space, which is substantially constant for incidences of between −45° and 45°.

The size of the interference pigment particles may be between 10 and 150 μm and better still between 10 and 50 μm. The term “size” denotes the size given by the statistical particle size distribution to half of the population, known as the D50.

Out of concern for aesthetics, it is preferable for the particles of the interference pigment not to be perceptible to the naked eye at the surface of the composition applied to its support. It is also desirable for the interference pigment particles not to have a size such that they give a sensation of discomfort on the skin. The use of particles less than or equal to 250 μm and better still less than or equal to 150 μm in size is thus preferred. The particle size may depend on the nature of the support onto which the composition is intended to be applied; certain parts of the body and of the face may, for example, tolerate more easily than others a larger size without causing discomfort.

The interference pigment may comprise at least one layer made of a material chosen from MgF₂, CeF₃, ZnS, ZnSe, Si, Ge, Te, SiO₂, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Fe₂O₃, Pt, Va, Ti, Ta, Zn, cryolite, alloys and polymers, and combinations thereof.

According to another of its aspects, a subject of the invention is also a composition comprising, in a physiologically acceptable medium, at least one interference pigment with a multilayer structure, the spectral reflectance of the composition and/or the pigment differing by not more than 20% from the reference spectral reflectance such as from a type of keratin material, for at least a portion of the visible spectrum 200 nm broad, this composition being characterized in that the multilayer structure comprises at least one layer substantially totally coating an underlying layer. By the phrase “substantially coating an underlying layer” it is not meant that each layer must totally coat the layer it covers or that any one layer completely covers any other layer, although this latter is preferred. It is preferred that through the use of a plurality of layers, the net effect is that the substrate and/or any coloring material in the core of the interference pigment will be totally coated. Moreover, the term does not require that all of the particles be completely coated. However, the majority should be completely coated and those that remain should not include a uncoated zones of rupture which characterize the particles described in WO 00/75240.

Such a composition may comprise an interference pigment with a multilayer structure as defined above.

Such a composition may also comprise a plurality of pigments having different structures, the pigments possibly being or not being all of interference type with a multilayer structure.

The composition may especially comprise a mixture of pigments, including at least one interference pigment, the proportions of the various pigments being chosen so as to obtain the spectral reflectance that is desired for the composition.

The composition may especially comprise a mixture of pigments, including at least one interference pigment, that makes it possible to obtain a spectral reflectance close to the reference spectral reflectance even in the case of variation of the refractive index of one phase of the composition, in order, for example, to take account of the absorption of sebum by the composition or the evaporation of an ingredient.

The multilayer structure is advantageously configured such that the composition is substantially non-goniochromatic for incidences of between −45° and 45°, which can allow the appearance of the skin to be recreated more faithfully.

For the purposes of the present invention, the expression “non-goniochromatic composition” denotes a composition that makes it possible to obtain, when it is spread onto a support, a colour trajectory in the a*, b* plane of the CIE 1976 calorimetric space which corresponds to a variation Dh of the hue angle h of not more than 20°, when the angle of observation is varied relative to the normal between 0° and 80°, for an angle of incidence of the light of 45°. The colour trajectory may be measured, for example, using an Instrument Systems brand spectrogonioreflectometer of reference GON 360 Goniometer, after the composition has been spread in fluid form to a thickness of 300 μm using an automatic spreader onto an Erichsen brand contrast card of reference TYP 24/5, the measurement being performed on the black background of the card. The lightness parameter L* may also be substantially constant for incidences ranging from 0° to 50°.

Preferably, the spectral reflectance of the composition applied to its support differs, for a given range of the spectrum, for example for a range at least 200 nm broad, for example for at least the range from 500 nm to 700 nm, by not more than 10% of the reference spectral reflectance and better still by not more than 5% or even better by not more than 2% when it is desired to optimally imitate the colour of the skin.

A subject of the invention is also the use of a composition as defined above to camouflage skin imperfections, especially wrinkles, spots, marks, rosacea, veins and blackheads.

A subject of the invention is also the use of a composition as defined above to attenuate a dominant chromatic of the skin, for example yellow.

A subject of the invention is also a process for manufacturing an interference pigment with a multilayer structure, characterized in that it comprises the following step: producing the multilayer structure such that it comprises at least one layer substantially totally coating an underlying layer and such that the spectral reflectance of the resulting interference pigment differs, for at least a portion of the visible spectrum 200 nm broad, by not more than 20% from the reference spectral reflectance, better still by not more than 10% and even better still by not more than 2%. This process can also include the step of pre-defining a reference spectral reflectance, preferably one having at least one portion that is substantially identical to the spectral reflectance of a desired type of keratin material. This reference can be used as a target upon which to base the desired properties of the interference pigment.

A subject of the invention is also a process for manufacturing a composition to be applied to the skin or the integuments, this composition comprising, in a physiologically acceptable medium, at least one interference pigment with a multilayer structure, characterized in that it comprises the following step:

producing the multilayer structure such that it comprises at least one layer substantially totally coating an underlying layer and such that the spectral reflectance of the composition, applied to its support, differs, for at least a portion of the visible spectrum 200 nm broad, by not more than 20% from the reference spectral reflectance, better still by not more than 10% and even better still by not more than 2%. This process can also include the step of pre-defining a reference spectral reflectance having at least one portion that is substantially identical to the spectral reflectance of a desired type of keratin material. This reference can be used as a target upon which to base the desired properties of the interference pigment. The resulting interference pigment can be mixed with a physiologically acceptable medium.

The reference spectral reflectance may be determined, for example, from information associated with the person intended to receive the composition, for example following a reply to a questionnaire or to a measurement taken on the skin or hair, (also referred to herein as various types of keratin material). It may also be determined experimentally or hypothetically by use of composite data taken from a plurality of questionnaires, measurements taken from keratin material, empirical measurements, calculations and the like. The reference spectral reflectance may also be set arbitrarily. However, the reference spectral reflectance will be selected from and/or based upon, a color range of human skin and/or hair (e.g. flesh tones and hair colors.) and even more preferably from keratin material and specifically skin.

### BRIEF DESCRIPTION OF THE DRAWINGS

The invention may be understood more clearly on reading the following detailed description of non-limiting working examples thereof, and on examining the attached drawing, in which:

FIG. 1 shows, in isolation and very schematically, an example of a particle with a multilayer structure in accordance with one working example of the invention,

FIG. 2 shows, in isolation and very schematically, another example of a particle with a multilayer structure according to the invention,

FIG. 3 shows, in isolation and very schematically, an example of a particle with a multilayer structure of the prior art, and

FIG. 4 shows the spectral reflectance of different types of skin, the percentage of reflected light being given on the y-axis and the wavelength in nm on the x-axis, and also the spectral reflectance of various cosmetic compositions produced in accordance with the invention.

### DETAILED DESCRIPTION

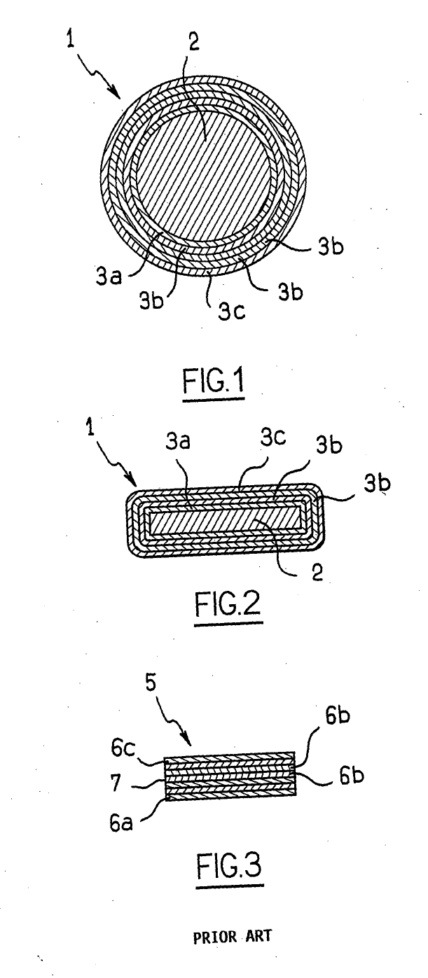

In FIGS. 1 to 3, the actual relative proportions of the various components have not been respected, out of concern for clarity of the drawing.

FIG. 1 shows a particle 1 of pigment comprising a substrate 2 surrounded with a plurality of concentric layers, i.e.:

an extreme inner layer 3a deposited on the substrate 2,

an extreme outer layer 3c, and

a plurality of intermediate layers 3b between the extreme layers 3a and 3c.

Out of concern for clarity of the drawing, only three intermediate layers 3b have been shown in FIG. 1. Needless to say, FIG. 1 is only one schematic representation, and the actual number of intermediate layers may be different without departing from the context of the present invention, the number of intermediate layers being dependent on the optical properties that are desired for the pigment and possibly being between 3 and 50, for example. Indeed, no intermediate layers may be needed at all.

In the example under consideration, the substrate 2 has a spherical shape, consisting, for example, of a solid glass microsphere.

It would not constitute a departure from the context of the present invention if the substrate had a shape other than spherical, for example a platelet form, as illustrated in FIG. 2. The expression “substrate in platelet form” denotes a substrate for which the ratio of the largest dimension to the thickness is greater than or equal to 5, or even 10 or 15. The thickness of a substrate in platelet form is, for example between about 0.5 μm and about 5 μm.

The substrate 2 may be mono-material or multi-material, and solid or hollow. The substrate 2 may be organic or mineral. The substrate may be natural, but a synthetic substrate is preferably used, which makes it possible to obtain a given form more easily. Although a substrate is preferred, no substrate is required.

The substrate may be chosen from glasses, ceramics, graphite, metal oxides, aluminas, silicas, silicates, especially aluminosilicates and borosilicates and synthetic mica, this list not being limiting.

FIG. 3 shows a particle 5 of pigment according to the prior art.

It may be seen that this particle comprises two extreme layers 6a and 6c and, between them, a plurality of intermediate layers 6b, the stacking of the various layers 6a, 6b and 6c taking place without one given layer entirely covering an adjacent layer and the various layers 6a, 6b and 6c not extending over the cut edge 7 of the particle resulting from rupture of the multiplayer film as described in WO 00/75240.

Returning to FIGS. 1 and 2, the various layers 3a, 3b and 3c may be deposited on the substrate 2 by means of a plant, not shown, comprising a chamber for exposing the particles to a material to be deposited, in which chamber the particles are placed in motion via a vibrating device. Such a plant is described in the article “Overcoated Microspheres for Specific Optical Powders” from the review “Applied Optics”, vol. 41, No. 6 of Jun. 1, 2002.

Needless to say, the process described in the said article is merely one example among other processes that may be used to deposit the various layers 3a, 3b and 3c of a pigment particle.

The refracted indices of the various layers 3a, 3b and 3c and the respective thicknesses thereof are chosen by applying the well known theory of interference filters, such that the spectral reflectance of the multilayer structure has the desired properties, under the conditions of use.

The multilayer structure may be formed from a succession of layers with high and low refractive indices, the layers with a high index comprising, for example, a metal oxide, and the layers with a low index comprising a metal.

It is possible to produce with a relatively small number of layers an interference pigment that makes it possible to produce a cosmetic or care composition whose spectral reflectance is relatively close to that of a predetermined type of skin, Caucasian or other, for example in order to produce a natural makeup.

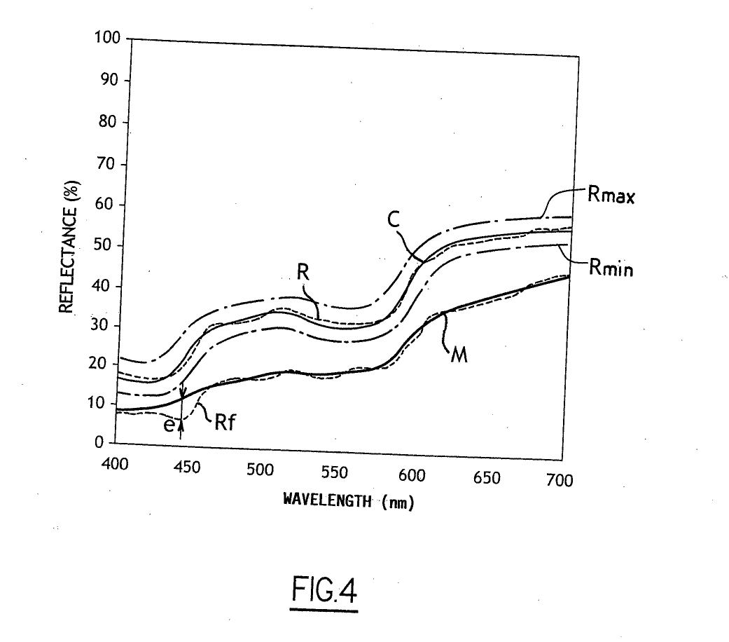

It should be noted that, in general, the optical properties of the skin are different depending on the origin of the individual and, by way of example, FIG. 4 shows the in vivo spectral reflectance of the skin for an individual of Caucasian type (curve C) and of half-caste type (curve M), as a function of the wavelength of the incident radiation. The measurements are taken using an integrating sphere placed on the skin. Reference may appropriately be made to the article “Spectral Reflectance of human skin in vivo” P. H. Andersen, P. Bjerring—Photodermatol—Photo imuno—Photomed 1990: 5-12.

If it is desired to optimally reproduce the spectral reflectance of a type of skin, for example Caucasian skin, the multilayer structure will be produced such that the spectral reflectance R of the composition that comprises the pigment is within minimum Rₘᵢₙ and maximum Rₘₐₓ limits, these curves Rₘᵢₙ and Rₘₐₓ not differing, for example, by more than 10% from the curve C over the entire visible spectrum, curve C serving in the example under consideration as the reference spectral reflectance.

In FIG. 4, it may thus been seen that the reflectance R of the composition extends in a “tunnel” delimited by the curves Rₘᵢₙ and Rₘₐₓ.

However, it would not constitute a departure from the context of the present invention if the reference spectral reflectance that it is desired to reproduce differed substantially from the spectral reflectance of a type of skin in a given portion of the visible spectrum, in order, for example, to correct certain dominant chromatics thereof.

The reference spectral reflectance may thus be chosen so as to have a reflectance that is smaller than that of the selected type of skin, for example in yellow.

FIG. 4 shows, for illustrative purposes, a reference spectral reflectance R_{f} that differs substantially from curve M for the portion of the spectrum between 450 and 500 nm, in order to attenuate the dominant yellow chromatic of a half-caste skin. The difference between the spectral reflectance of the composition and curve M may be, for example, greater than about 5% for at least one value of the spectrum.

A interference pigment with a multilayer structure according to the invention may be incorporated into the formulation of a large number of compositions comprising a physiologically acceptable medium and intended, for example, for making up body or facial skin.

The interference pigment can represent between 0.01% and 50% and preferably between 0.5% and 25% by weight relative to the total weight of the composition.

The physiologically acceptable medium will be adapted to the nature of the support onto which the composition is to be applied (skin and/or hair), and also the form in which the composition is intended to be packaged, especially solid or fluid at room temperature and atmospheric pressure.

The composition may especially comprise a physiologically acceptable medium which is an aqueous cosmetic medium and/or a fatty phase.

The composition may comprise a physiologically acceptable medium which is water or a mixture of water and of hydrophilic organic solvent, for example an alcohol.

The composition may also comprise a physiologically acceptable medium which includes fillers. The term “filler” denotes particles of any form, which are insoluble in the medium of the composition, irrespective of the temperature at which the composition is manufactured. These fillers may serve especially to modify the rheology or the texture of the composition. The nature and amount of the solid substances will depend on the desired mechanical properties and textures. Examples of fillers that may be mentioned, inter alia, include talc, mica, silica, kaolin and polyamide powders, for example Nylon® or Orgasol® powders.

The composition may also contain one or more cosmetic, dermatological, hygiene or pharmaceutical active agents.

As active agents that may be used in the composition, mention may be made of moisturizers (polyol, for instance glycerol), vitamins (C, A, E, F, B or PP), essential fatty acids, ceramides, sphingolipids or liposoluble sunscreens or in the form of nanoparticles, and specific active agents for treating the skin (protective, antibacterial, antiwrinkle, etc. agents). These active agents may be used, for example, at concentrations of from 0 to 20% and especially from 0.001% to 15% relative to the total weight of the composition.

The cosmetic composition may also contain a physiologically acceptable medium which includes ingredients commonly used in cosmetics, for instance thickeners, surfactants, trace elements, moisturizers, softeners, sequestering agents, fragrances, acidifying or basifying agents, preserving agents, antioxidants and UV-screening agents, or mixtures thereof.

Depending on the envisaged type of application, the cosmetic composition may furthermore comprise a physiologically acceptable medium which includes constituents conventionally used in the fields under consideration, which are present in an amount that is suitable for the desired presentation form.

The cosmetic composition may be in any presentation form normally used for topical application and especially in an anhydrous form, in the form of an oily or aqueous solution, an oily or aqueous gel, an oil-in-water or water-in-oil emulsion, a multiple emulsion, or a dispersion of oil in water by means of vesicles located at the oil/water interface.

The composition of the invention may be in the form of a powder, a liquid, a solid or a semi-solid, especially a product cast as a stick or in a dish, a tube, a paste or a more or less fluid cream.

The composition may constitute, inter alia, a solid or fluid foundation, a concealer product or an eye contour product, a body makeup product or an antisun product or skin treatment product.

The composition of the invention may be obtained according to the preparation processes conventionally used.

The example given below is presented as a non-limiting illustration of the invention.

A foundation intended to approach the colour of Caucasian skin, having the formulation below, was prepared by mixing together in a conventional manner:

| | | |
|---|---|---|
| Polydimethylorganosiloxane crosslinked in | 20 | g |
| polydimethylsiloxane 6 cs | | |
| (gelling and matting agent) | | |
| Cyclopentadimethylsiloxane (oil) | 29 | g |
| Hydrogenated isoparaffin (oil) | 10 | g |
| Talc (filler) | 6 | g |
| Interference pigment | 10 | g |
| Modified hectorite (gelling clay) | qs 100 | g |

The interference pigment has, for example, the following structure:

| | | REFRACTIVE | |
|---|---|---|---|
| LAYER | MATERIAL | INDEX n | THICKNESS (nm) |
| 1 (outermost) | Al₂O₃ | 1.6726 | 92.1 |
| 2 | Au | 0.7382 | 23.6 |
| 3 | Al₂O₃ | 1.6726 | 81.4 |
| 4 | Au | 0.7382 | 1.8 |
| 5 | Al₂O₃ | 1.6726 | 76.0 |
| 6 | Au | 0.7382 | 13.6 |
| 7 | Al₂O₃ | 1.6726 | 94.6 |
| 8 | Au | 0.7382 | 12.6 |
| SUBSTRATE | glass microbead | 1.5 | |
| | 10 μm diameter | | |

The invention makes it possible to produce compositions with a high covering power while at the same time giving the made-up skin, for example of Caucasian type, a natural appearance, making it difficult to detect the presence of the composition.

It is possible not only to vary the spectral reflectance but also the spectral transmittance, by suitably selecting the various layers of the multilayer structure of the pigment.

The invention makes it possible to produce different colours by means of a combination of pigments including at least one interference pigment that differ from each other only in the number and thicknesses of the layers of the multilayer structure. The formulation of the cosmetic and/or dermatological compositions is thereby found to be easier to establish for a range of shades than with the pigments of the prior art.

The interference pigments or mixture of pigments that include at least one interference pigment can be produced from materials selected to provide the desired spectral reflectance, one that is close to the appropriate reference spectral reflectance, even in the case of variation of the refractive index of one phase of the composition. Such changes in refractive index may occur due to, for example, the absorption of sebum by the composition, the evaporation of an ingredient, temperature, weather and the like. This can be accomplished by selection of substrates, pigments and layers that will be relatively insensitive to such changes in their immediate environment. This may all be tested experimentally.

Needless to say, the invention is not limited to the examples that have just been described. In particular, the composition may be produced tailor-made, if necessary, after having measured the spectral reflectance of the skin of the individual onto which it is intended to be applied. In this case, the multilayer structure is calculated such that the composition incorporating the pigment optimally approaches the reflectance of the skin of the individual, or even differs therefrom for certain values of the spectrum in order to correct a dominant chromatic.

Throughout the description, including the claims, the expression “comprising one” should be understood as being synonymous with “comprising at least one”, unless otherwise specified.

1. An interference pigment comprising: a multilayer structure with a spectral reflectance differing by not more than about 20% from a reference spectral reflectance for at least a portion of the visible spectrum 200 nm broad, said multilayer structure including at least one layer substantially totally coating an underlying layer

2. The pigment according to claim 1, wherein said pigment is in the form of particles having a globular general form.

3. The pigment according to claim 2, wherein said particles have a spherical form.

4. The pigment according to claim 2, wherein said particles comprise a substrate having a globular form, on which are deposited various layers of said multilayer structure.

5. The pigment according to claim 4, wherein said substrate has a spherical form.

6. The pigment according to claim 4, wherein said substrate comprises at least one microsphere.

7. The pigment according to claim 4, wherein said substrate is made of glass or metal.

8. The pigment according to claim 2, wherein said particles have a symmetrical form relative to a centre of symmetry.

9. The pigment according to claim 1, wherein said spectral reflectance of said interference pigment differs, for at least one range 200 nm broad, by not more than about 10% from said reference spectral reflectance.

10. The pigment according to claim 9, wherein said spectral reflectance of said interference pigment differs, for at least one range 200 nm broad, from said reference spectral reflectance by not more than about 2%.

11. The pigment according to claim 1, wherein said spectral reflectance of said interference pigment differs, over at least the 500-700 nm range, by not more than about 10% from said reference spectral reflectance.

12. The pigment according to claim 11, wherein said spectral reflectance of said interference pigment differs, over at least the 500-700 nm range, from said reference spectral reflectance of by not more than about 2%.

13. The pigment according to claim 1, having a lightness value L*, measured in the CIE 1976 space, which is substantially constant for incidences of between −45° and 45°.

14. The pigment according to claim 2, wherein said particles have a size lying between about 10 and about 150 μm.

15. The pigment according to claim 14, wherein said size of the particles lies between about 10 and about 50 μm.

16. The pigment according to claim 1, wherein said reference spectral reflectance is human skin.

17. A composition, comprising: a physiologically acceptable medium and mixed therein at least one interference pigment having a multilayer structure with a spectral reflectance differing by not more than about 20% from a reference spectral reflectance for at least a portion of the visible spectrum 200 nm broad, said multilayer structure including at least one layer substantially totally coating an underlying layer.

18. The composition according to claim 17, wherein said composition is substantially non-goniochromatic.

19. The composition according to claim 17, wherein said spectral reflectance of said interference pigment differs by not more than about 20% from said reference spectral reflectance over the entire visible spectrum.

20. The composition according to claim 17, further comprising a mixture of pigments including at least one interference pigment, the proportions of the various pigments being chosen so as to obtain the desired spectral reflectance for the composition.

21. The composition according to claim 17, wherein said interference pigment maintains a spectral reflectance that is close to said reference spectral reflectance even in the case of variation of the refractive index.

22. The composition according to claim 17, wherein the multilayer structure is configured such that said composition is substantially non-goniochromatic for incidences of between −45° and 45°.

23. Composition according to claim 17, wherein said spectral reflectance of the composition applied to its support differs, for a given range of the spectrum at least 200 nm broad, by not more than about 10% from the reference spectral reflectance.

24. Composition according to claim 23, wherein the spectral reflectance of the composition applied to its support differs, for a given range of the spectrum at least 200 nm broad, by not more than about 5% from the reference spectral reflectance.

25. Composition according to claim 24, wherein the spectral reflectance of the composition applied to its support differs, for a given range of the spectrum at least 200 nm broad, by not more than about 2% from the reference spectral reflectance.

26. Composition according to claim 17, wherein the spectral reflectance of the composition applied to its support differs, for the range of the spectrum from 500 nm to 700 nm, by not more than about 10% from the reference spectral reflectance.

27. Composition according to claim 26, wherein the spectral reflectance of the composition applied to its support differs, for the range of the spectrum from 500 to 700 nm, by not more than about 5% from the reference spectral reflectance.

28. Composition according to claim 27, wherein the spectral reflectance of the composition applied to its support differs, for the range of the spectrum from 500 to 700 nm, by not more than about 2% from the reference spectral reflectance.

29. A method for camouflaging a zone of skin, comprising:
applying on the zone of the skin a composition as defined is claim 17.

30. The method according to claim 29, wherein said zone of said skin comprises a skin imperfection chosen in the group consisting of: wrinkles, spots, marks, rosacea, veins and blackheads.

31. A method for attenuating a dominant chromatic of a zone of skin, comprising:
applying on said zone of the skin a composition as defined in claim 17.

32. The method according to claim 31, wherein the dominant chromatic that is attenuated is yellow.

33. A process for manufacturing an interference pigment with a multilayer structure, comprising the steps of:
defining a reference spectral reflectance of a type of keratin material,
producing the multilayer structure such that it comprises at least one layer substantially totally coating an underlying layer and such that a spectral reflectance of said pigment differs, for at least a portion of the visible spectrum 200 nm broad, by not more than about 20% from said reference spectral reflectance.

34. The process according to claim 33, wherein said spectral reflectance of said pigment differs by not more than about 10% from said reference spectral reflectance, for at least a portion of the visible spectrum 200 nm broad.

35. The process according to claim 34, wherein said spectral reflectance of said pigment differs by not more than 2% from said reference spectral reflectance, for at least a portion of the visible spectrum 200 nm broad.

36. A process for manufacturing a composition to be applied to the skin or the integuments, comprising the steps of:
defining a reference spectral reflectance having a spectral reflectance of a type of keratin material;
producing the multilayer structure such that it comprises at least one layer substantially totally coating an underlying layer and such that a spectral reflectance of said composition, applied to its support, differs, for at least a portion of the visible spectrum 200 nm broad, by not more than about 20% from said reference spectral reflectance;
and mixing in a physiologically acceptable medium and at least one interference pigment with said multiplayer structure.

37. The process according to claim 36, wherein said spectral reflectance of said composition applied to its support differs by not more than about 10% from said reference spectral reflectance, for at least a portion of the visible spectrum 200 nm broad.

38. The process according to claim 36, wherein said spectral reflectance of said composition applied to its support differs by not more than about 2% from said reference spectral reflectance, for at least a portion of the visible spectrum 200 nm broad.

39. The process according to claim 36, wherein said reference spectral reflectance is determined from information associated with a person who is intended to receive said composition.

40. The process according to claim 36, wherein said reference spectral reflectance is determined from a measurement made on the skin.
